(19) United States
(12) Patent Application Publication
(10) Pub. No.: US 2023/0309964 A1
(43) Pub. Date: Oct. 5, 2023

(54) TRANSDUCER WITH ROTATABLE HEAD AND RECONFIGURABLE ARRAY CURVATURE

(71) Applicant: SHENZHEN MINDRAY BIO-MEDICAL ELECTRONICS CO., LTD., Shenzhen (CN)

(72) Inventors: Glen W. McLAUGHLIN, San Carlos, CA (US); Satchi PANDA, Fremont, CA (US)

(21) Appl. No.: 17/710,069

(22) Filed: Mar. 31, 2022

(51) Int. Cl. A61B 8/00

(52) U.S. Cl. CPC A61B 8/4466; A61B 8/4494; A61B 8/4461

(57) ABSTRACT

An ultrasound transducer device includes a handle portion, a transducer head, and a connector coupled to the transducer head and the handle portion to enable the transducer head to rotate with respect to the handle portion to provide different form factors for the ultrasound transducer device.

### TECHNICAL FIELD

This disclosure is generally related to ultrasound transducer devices, and more specifically to ultrasound transducer devices with a rotatable head and reconfigurable array curvature.

### BACKGROUND

Ultrasound is routinely used to scan multitude of human anatomies both externally and intra-operatively. In practice, an ultrasound transducer device is employed for this purpose. At times, an anatomy being scanned or the procedure being performed demands certain geometric form factor of the ultrasound transducer to enable proper access, maneuverability, and ergonomics, and to provide a necessary field of view with an acceptable diagnostic image quality. To achieve this goal, a plurality of same ultrasound stacks/probes are packaged into different form factors creating separate, distinct transducers. This unnecessarily increases the number of ultrasound probes a clinic needs to carry.

### SUMMARY

Described herein are ultrasound transducer devices with a rotatable head and reconfigurable array curvature that can provide flexible form factors to fit various requirements for ultrasonic scanning.

In one aspect, an ultrasound transducer device is provided. The ultrasound transducer device includes a handle portion, a transducer head, and a connector coupled to the transducer head and the handle portion to enable the transducer head to rotate with respect to the handle portion to provide different form factors for the ultrasound transducer device.

In some embodiments, the connector includes a shaft. In some embodiments, the connector includes a rotating mechanism such as a ball and socket joint. The connector allows electrical connection between the transducer head and the handle portion.

In some embodiments, the transducer head includes a substrate and an array of ultrasound transducers disposed on the substrate.

In some embodiments, the ultrasound transducers are disposed on the substrate in one dimension. In some embodiments, the ultrasound transducers are disposed on the substrate in two dimensions.

In some embodiments, the substrate includes a flat surface, a convex surface or a concave surface such that the array of ultrasound transducers is configured as a flat array, a convex array, or a concave array.

In some embodiments, the substrate is deformable such that the array of ultrasound transducers is switched among the flat array, the convex array, and the concave array.

In some embodiments, the connector includes a sliding head to enable the transducer head to slide with respect to the handle portion. In some embodiments, the transducer head includes a trench coupled to the sliding head of the connector such that the sliding head slides in the trench.

In another aspect, an ultrasound transducer device is provided. The ultrasound transducer device includes a handle portion, a transducer head, and a connector coupled to the transducer head and the handle portion to enable the transducer head to rotate with respect to the handle portion to provide different form factors for the ultrasound transducer device. The transducer head includes a flexible substrate and an array of ultrasound transducers disposed on the flexible substrate.

In some embodiments, the ultrasound transducers are disposed on the flexible substrate in one dimension. In some embodiments, the ultrasound transducers are disposed on the flexible substrate in two dimensions.

In some embodiments, the flexible substrate comprises a flat surface, a convex surface or a concave surface such that the array of ultrasound transducers is configured as a flat array, a convex array, or a concave array.

In some embodiments, the flexible substrate is deformed such that the array of ultrasound transducers is switched among the flat array, the convex array, and the concave array.

In yet another aspect, an ultrasound transducer device is provided. The ultrasound transducer device includes a handle portion, a transducer head, and a connector coupled to the transducer head and the handle portion to enable the transducer head to rotate with respect to the handle portion to provide different form factors for the ultrasound transducer device. The transducer head includes a flexible substrate and an array of ultrasound transducers disposed on the flexible substrate. The transducer head further includes a motor and a link. The link is coupled to the motor and connected to the flexible substrate. The motor drives the link to deform the flexible substrate and change the curvature of the array of ultrasound transducers.

In yet another aspect, an ultrasound machine is provided. The ultrasound machine includes an ultrasound transducer device, a processor, and a display device. The ultrasound transducer device is configured to transmit ultrasound signals to an object and receive echo signals from the object. The ultrasound transducer device includes a handle portion, a transducer head, and a connector coupled to the transducer head and the handle portion to enable the transducer head to rotate with respect to the handle portion to provide different form factors for the ultrasound transducer device. The processor configured to convert the echo signals into an image. The display device configured to display the image.

### BRIEF DESCRIPTION OF THE DRAWINGS

Certain features of various embodiments of the present technology are set forth with particularity in the appended claims. A better understanding of the features and advantages of the technology will be obtained by reference to the following detailed description that sets forth illustrative embodiments, in which the principles of the disclosure are utilized, and the accompanying drawings of which:

FIG. 1A is a diagram illustrating an ultrasound transducer device, according to one example embodiment.

FIG. 1B a diagram illustrating another configuration of the ultrasound transducer device illustrated in FIG. 1A, according to one example embodiment.

FIG. 1C shows a configuration of the ultrasound transducer device illustrated in FIG. 1A, where the transducer head can slide with respect to the handle portion, according to one example embodiment.

FIG. 1D illustrates a configuration of an ultrasound transducer device, where the transducer head is rotated with respect to the handle portion by 90 degrees counterclockwise from the configuration shown in FIG. 1C.

FIG. 1E illustrates another configuration of an ultrasound transducer device, where the transducer head is rotated with respect to the handle portion by about 45 degrees counterclockwise from the configuration shown in FIG. 1C.

FIG. 2 is a diagram illustrating a cross-sectional view of an ultrasound transducer device, according to one example embodiment.

FIG. 3 is a diagram illustrating a cross-sectional view of another ultrasound transducer device, according to one example embodiment.

FIG. 4 is a diagram illustrating a configuration of an array of ultrasound transducers of a transducer head, according to one example embodiment.

FIG. 5 is a diagram illustrating a configuration of another array of ultrasound transducers of a transducer head, according to one example embodiment.

FIG. 6 is a diagram illustrating a configuration of yet another array of ultrasound transducers of a transducer head, according to one example embodiment.

FIG. 7A is a diagram illustrating a configuration of an ultrasound transducer device, according to one example embodiment.

FIG. 7B shows another configuration of an ultrasound transducer device, where the transducer head is rotated by 90 degrees counterclockwise with respect to the handle portion from the configuration illustrated in FIG. 7A.

FIG. 8A is a diagram illustrating a configuration of another ultrasound transducer device, according to one example embodiment.

FIG. 8B shows another configuration of an ultrasound transducer device, where the transducer head is rotated by 90 degrees from the plane parallel to the paper to the normal direction of the paper, from the configuration illustrated in FIG. 8A.

FIG. 9A is a diagram illustrating a cross-sectional view of a transducer head, according to one example embodiment.

FIG. 9B is a diagram illustrating a cross-sectional view of a transducer head, wherein the flexible substrate is deformed to a concave shape, according to one example embodiment.

FIG. 9C is a diagram illustrating a cross-sectional view of a transducer head, wherein the flexible substrate is deformed to a convex shape, according to one example embodiment.

FIG. 10 is a diagram illustrating an ultrasound machine, according to one example embodiment.

### DETAILED DESCRIPTION OF EMBODIMENTS

In the following description, certain specific details are set forth in order to provide a thorough understanding of various embodiments of the disclosure. However, one skilled in the art will understand that the disclosure may be practiced without these details. Moreover, while various embodiments of the disclosure are disclosed herein, many adaptations and modifications may be made within the scope of the disclosure in accordance with the common general knowledge of those skilled in this art. Such modifications include the substitution of known equivalents for any aspect of the disclosure in order to achieve the same result in substantially the same way.

Unless the context requires otherwise, throughout the present specification and claims, the word “comprise” and variations thereof, such as, “comprises” and “comprising” are to be construed in an open, inclusive sense, that is as “including, but not limited to.” Recitation of numeric ranges of values throughout the specification is intended to serve as a shorthand notation of referring individually to each separate value falling within the range inclusive of the values defining the range, and each separate value is incorporated in the specification as it were individually recited herein. Additionally, the singular forms “a,” “an” and “the” include plural referents unless the context clearly dictates otherwise.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment, but may be in some instances. Furthermore, the particular features, structures, or characteristics may be combined in any suitable manner in one or more embodiments.

Various embodiments described herein are directed to various ultrasound transducer devices that include a rotatable head and reconfigurable array curvature to provide flexible form factors to fit various requirements for ultrasound examinations. In one embodiment, an ultrasound transducer device includes a handle portion, a transducer head, and a connector coupled to the transducer head and the handle portion to enable the transducer head to rotate with respect to the handle portion to provide different form factors for the ultrasound transducer device.

In another embodiment, an ultrasound transducer device includes a handle portion, a transducer head, and a connector coupled to the transducer head and the handle portion to enable the transducer head to rotate with respect to the handle portion to provide different form factors for the ultrasound transducer device. The transducer head includes a flexible substrate and an array of ultrasound transducers disposed on the flexible substrate.

In yet another embodiment, an ultrasound transducer device includes a handle portion, a transducer head, and a connector coupled to the transducer head and the handle portion to enable the transducer head to rotate with respect to the handle portion to provide different form factors for the ultrasound transducer device. The transducer head includes a flexible substrate and an array of ultrasound transducers disposed on the flexible substrate. The transducer head further includes a motor and a link. The link is coupled to the motor and connected to the flexible substrate. The motor drives the link to deform the flexible substrate and change the curvature of the array of ultrasound transducers.

The disclosed techniques provide a various single transducer device that can be reconfigurable to achieve different form factors to increase application coverage and use cases.

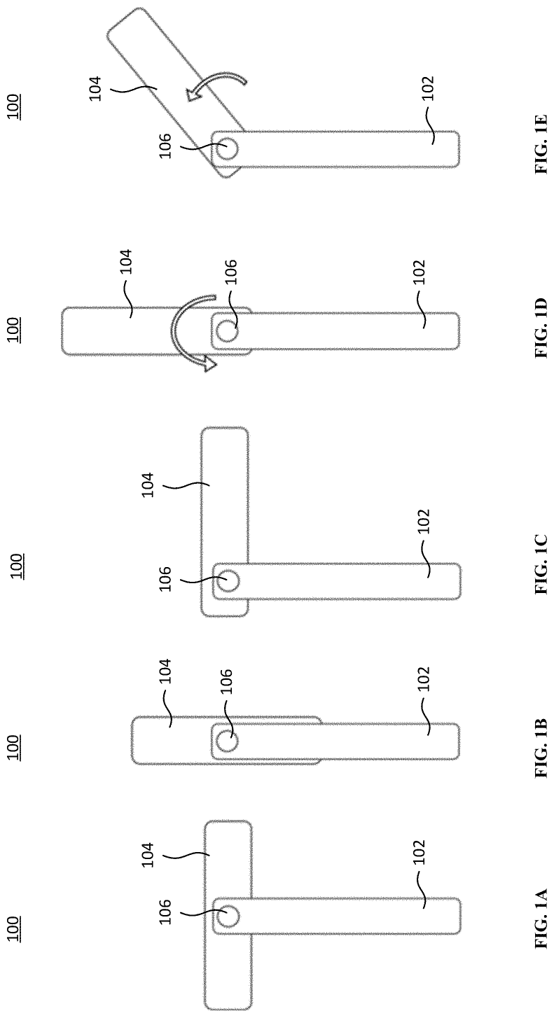

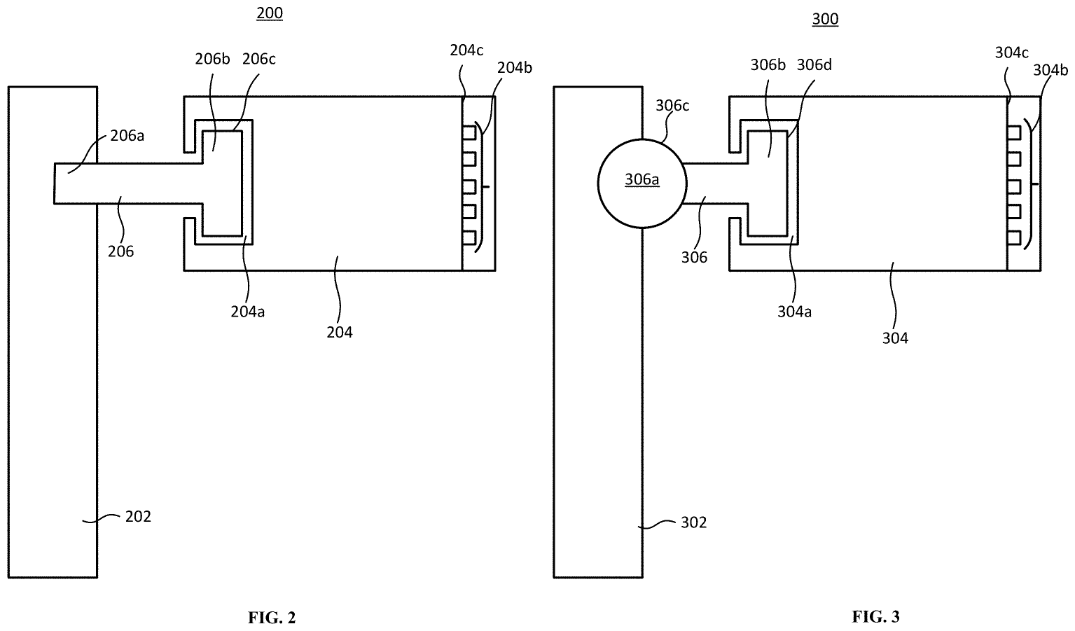

Embodiments will now be explained with accompanying figures. Reference is first made to FIG. 1A. FIG. 1A is a diagram illustrating an ultrasound transducer device 100, according to one example embodiment. The ultrasound transducer device 100 includes a handle portion 102, a transducer head 104, and a connector 106 that connects the handle portion 102 and transducer head 104 together. The connector 106 enables the transducer head 104 to rotate with respect to the handle portion 102 to provide different form factors for the ultrasound transducer device 100. In some embodiments, the connector 106 is a shaft that has a first end portion fixed on the handle portion 102 and a second end portion coupled to the transducer head 104 to allow the transducer head 104 to rotate.

As shown in FIG. 1A, the transducer head 104 and the handle portion 102 forms a T shape, that is, the transducer head 104 is extended in a direction perpendicular to a direction in which the handle portion 102 is extended. FIG. 1B shows another configuration of the ultrasound transducer device 100, where the transducer head 104 and the handle portion 102 forms an I shape. In the configuration shown in FIG. 1B, the transducer head 104 is extended in a direction in parallel with a direction in which the handle portion 102 is extended. This can be understood as a transformed configuration from that shown in FIG. 1A, in which the transducer head 104 is rotated by 90 degrees clockwise or counterclockwise. The rotation degree is not limited to this example. Any other rotation degree can be implemented.

In some embodiments, additional or alternative to rotation, the connector 106 enables the transducer head 104 to slide with respect to the handle portion 102. The connector 106 may include a sliding mechanism (as will be explained below) to allow the transducer head 104 to slide with respect to the handle portion 102 to provide more form factors for various applications. For example, FIG. 1C shows a configuration of the ultrasound transducer device 100, where the transducer head 104 is slid with respect to the handle portion 102 from the configuration shown in FIG. 1A. In this example, the transducer head 104 is slid to from its middle portion to the left portion.

In some embodiments, after the transducer head 104 is slid with respect to the handle portion 102, the transducer head 104 can be rotated with respect to the handle portion 102. FIG. 1D illustrates a configuration of the ultrasound transducer device 100, where the transducer head 104 is rotated with respect to the handle portion 102 by 90 degrees counterclockwise from the configuration shown in FIG. 1C. FIG. 1E illustrates another configuration of the ultrasound transducer device 100, where the transducer head 104 is rotated with respect to the handle portion 102 by about 45 degrees counterclockwise from the configuration shown in FIG. 1C. It should be noted that the examples shown in FIGS. 1A-1E do not limit the scope of the disclosure. The transducer head 104 can be rotated with respect to the handle portion 102 by any degree to accommodate an ultrasound examination. Further, the transducer head 104 can be slid with respect to the handle portion 102 to any position to accommodate an ultrasound examination. In some embodiments, the transducer head 104 can be rotated with respect to the handle portion 102 and then slid with respect to the handle portion 102. In some embodiments, the transducer head 104 can be slid with respect to the handle portion 102 and then rotated with respect to the handle portion 102.

FIG. 2 is a diagram illustrating a cross-sectional view of an ultrasound transducer device 200, according to one example embodiment. The ultrasound transducer device 200 includes a handle portion 202, a transducer head 204, and a connector 206 coupled between the handle portion 202 and the transducer head 204. The connector 206 has a first end portion 206a coupled to the handle portion 202 and a second end portion 206b coupled to the transducer head 204. The first end portion 206a of the connector 206 enables the transducer head 204 to rotate with respect to the handle portion 202. The second end portion 206b of the connector 206 enables the transducer head 204 to slide with respect to the handle portion 202. In the illustrated embodiment of FIG. 2, the second end portion 206b of the connector 206 includes a sliding head 206c while the transducer head 204 includes a sliding trench 204a that receives the sliding head 206c such that the sliding head 206c can move along the sliding trench 204a. Other sliding mechanisms are contemplated and can be applied to the ultrasound transducer device 200.

The transducer head 204 also includes an array of ultrasound transducers 204b disposed on a substrate 204c. Each of the ultrasound transducers 204b may be a piezoelectric transducer, a capacitive micromachined ultrasound transducer (cMUT), or a piezoelectric micromachined ultrasound transducers (pMUT). The substrate 204c may be rigid or flexible. In some embodiments, the substrate 204c may be a flexible printed circuit board or a ceramic board.

FIG. 3 is a diagram illustrating a cross-sectional view of another ultrasound transducer device 300, according to one example embodiment. The ultrasound transducer device 300 includes a handle portion 302, a transducer head 304, and a connector 306 coupled between the handle portion 302 and the transducer head 304. The connector 306 has a first end portion 306a coupled to the handle portion 302 and a second end portion 306b coupled to the transducer head 304. The first end portion 306a of the connector 306 enables the transducer head 304 to rotate with respect to the handle portion 302. The first end portion 306a includes a rotating mechanism 306c to enable the connector 306 to rotate with respect to the handle portion 302 to provide more flexibility to operate the ultrasound transducer device 300. In some embodiments, the rotating mechanism 306c may include a ball and socket joint, a ball bearing, etc.

The second end portion 306b of the connector 306 enables the transducer head 304 to slide with respect to the handle portion 302. In the illustrated embodiment of FIG. 3, the second end portion 306b of the connector 306 includes a sliding head 306d while the transducer head 304 includes a sliding trench 304a that receives the sliding head 306d such that the sliding head 306d can move along the sliding trench 304a. Other sliding mechanisms are contemplated and can be applied to the ultrasound transducer device 300.

The transducer head 304 also includes an array of ultrasound transducers 304b disposed on a substrate 304c. Each of the ultrasound transducers 304b may be a piezoelectric transducer, a cMUT, or a pMUT. The substrate 304c may be rigid or flexible. In some embodiments, the substrate 304c may be a flexible printed circuit board or a ceramic board.

The rotation and sliding actions of each of the ultrasound transducer devices 100-300 can be controlled by a user with a hand, or by a user selecting a pre-programmed rotation mode or a pre-programmed sliding mode from a user interface associated with the ultrasound transducer devices 100-300. In some embodiments, the rotation and sliding actions of each of the ultrasound transducer devices 100-300 can be controlled by a user inputting specific rotation and/or sliding degrees a user interface associated with the ultrasound transducer devices 100-300.

In some embodiments, the status of the rotation and sliding actions (e.g., a form factor) of each of the ultrasound transducer devices 100-300 can be communicated from the ultrasound transducer devices 100-300 to a central controller that controls a scanning operation or operation-assistant equipment.

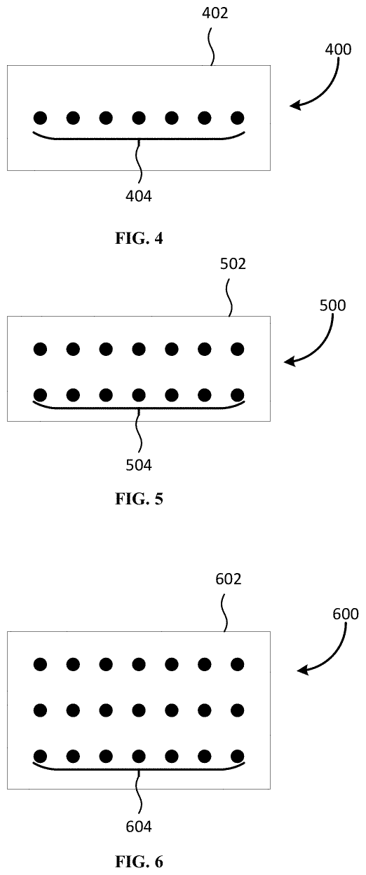

FIG. 4 is a diagram illustrating a configuration of an array of ultrasound transducers of a transducer head 400, according to one example embodiment. The ultrasound transducer head 400 may be any one of the transducer heads 104, 204, and 304 of FIGS. 1A-3. The ultrasound transducer head 400 includes a substrate 402 and an array of ultrasound transducers 404. As shown in FIG. 4, the ultrasound transducers 404 are disposed on the substrate 402 in one dimension (e.g., aligned in a line). The substrate 402 may be flexible or deformable. In some embodiments, the substrate 402 may be a flat surface, a convex surface, or a concave surface such that the array of ultrasound transducers 404 is configured as a flat array, a convex array, or a concave array. This allows the a transducer head 400 to accommodate different use cases. For example, when an anatomy to be scanned requires a certain field of view, compromise must be made to resolution and penetration to achieve a target field of view (FOV). With a flexible array, a radiologist can do a survey of the anatomy with the convex shape with a larger FOV, and subsequently, regions that are of specific interest can be scanned with a linear/flat or even concave shape that has a smaller FOV with improved resolution and penetration. This not only reduces a number of transducers a clinic needs to carry, but also provides both superior image quality and superior field of view during a single ultrasound scan using a single transducer device.

In some embodiments, the substrate 402 is deformed such that the array of ultrasound transducers 402 is switched among the flat array, the convex array, and the concave array.

FIG. 5 is a diagram illustrating a configuration of an array of ultrasound transducers of a transducer head 500, according to one example embodiment. The ultrasound transducer head 500 may be any one of the transducer heads 104, 204, and 304 of FIGS. 1A-3. The ultrasound transducer head 500 includes a substrate 502 and an array of ultrasound transducers 504. As shown in FIG. 5, the ultrasound transducers 504 are disposed on the substrate 502 in two dimensions (e.g., arranged in a matrix). The substrate 502 may be flexible or deformable. In some embodiments, the substrate 502 may be a flat surface, a convex surface, or a concave surface such that the array of ultrasound transducers 504 is configured as a flat array, a convex array, or a concave array. In some embodiments, the substrate 502 is deformed such that the array of ultrasound transducers 502 is switched among the flat array, the convex array, and the concave array.

FIG. 6 is a diagram illustrating a configuration of an array of ultrasound transducers of a transducer head 600, according to one example embodiment. The ultrasound transducer head 600 may be any one of the transducer heads 104, 204, and 304 of FIGS. 1A-3. The ultrasound transducer head 600 include a substrate 602 and an array of ultrasound transducers 604. The ultrasound transducer head 600 is similar to the ultrasound transducer head 500 illustrated in FIG. 5 but has more ultrasound transducers 604.

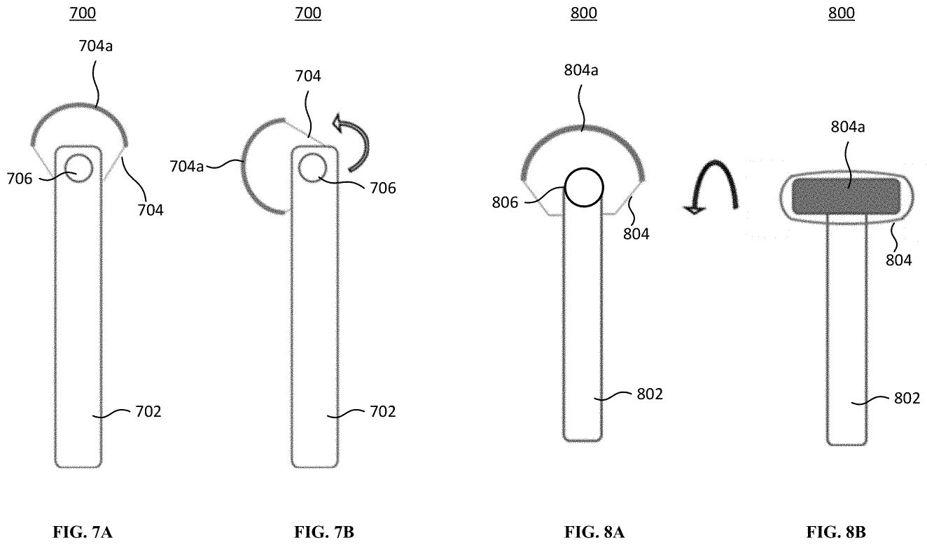

FIG. 7A is a diagram illustrating a configuration of an ultrasound transducer device 700, according to one example embodiment. The ultrasound transducer device 700 includes a handle portion 702, a transducer head 704, and a connector 706 that connects the handle portion 702 and transducer head 704 together. The transducer head 704 includes an array 704a of ultrasound transducers in a convex shape. The array 704a of ultrasound transducers is disposed on an outer end portion of the transducer head 704. The connector 706 enables the transducer head 704 to rotate with respect to the handle portion 702 to provide different form factors for the ultrasound transducer device 700. In some embodiments, the connector 706 is a shaft that has a first end portion fixed on the handle portion 702 and a second end portion coupled to the transducer head 704 to allow the transducer head 704 to rotate.

FIG. 7B shows another configuration of the ultrasound transducer device 700, where the transducer head 704 is rotated by 90 degrees counterclockwise with respect to the handle portion 702 from the configuration illustrated in FIG. 7A. The rotation degree is not limited to this example. Any other rotation degree can be implemented with the disclosed ultrasound transducer device. Although not shown in FIGS. 7A and 7B, the array 704a of ultrasound transducers may be in other shapes, such as a concave shape or a flat shape.

FIG. 8A is a diagram illustrating a configuration of another ultrasound transducer device 800, according to one example embodiment. The ultrasound transducer device 800 includes a handle portion 802, a transducer head 804, and a connector 806 that connects the handle portion 802 and transducer head 804 together. The transducer head 804 includes an array 804a of ultrasound transducers in a convex shape. The array 804a of ultrasound transducers is disposed on an outer end portion of the transducer head 804. The connector 806 enables the transducer head 804 to rotate with respect to the handle portion 802 to provide different form factors for the ultrasound transducer device 800. In some embodiments, the connector 806 is a rotating mechanism such as a ball and socket joint that has a first end portion fixed on the handle portion 802 and a second end portion coupled to the transducer head 804 to allow the transducer head 804 to rotate.

FIG. 8B shows another configuration of the ultrasound transducer device 800, where the transducer head 804 is rotated by 90 degrees from the plane parallel to the paper to the normal direction of the paper, from the configuration illustrated in FIG. 8A. The rotation degree is not limited to this example. Any other rotation degree can be implemented with the disclosed ultrasound transducer device. Although not shown in FIGS. 8A and 8B, the array 804a of ultrasound transducers may be in other shapes, such as a concave shape or a flat shape.

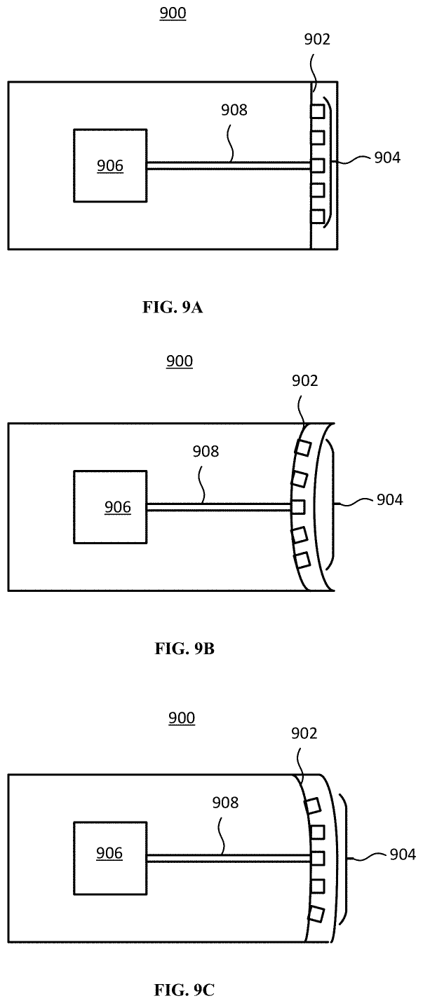

FIG. 9A is a diagram illustrating a cross-sectional view of a transducer head 900, according to one example embodiment. The transducer head 900 can be any one of the transducer head 104-304 illustrated in FIGS. 1A-3. The transducer head 900 includes a flexible substrate 902 and an array of ultrasound transducer 904 disposed on the substrate 902. The flexible substrate 902 illustrated in FIG. 9A is in a flat shape. The array of the ultrasound transducer 904 may be in one dimension (similar to that illustrated in FIG. 4) or in two dimensions (similar to that illustrated in FIGS. 5 and 6). The transducer head 900 further includes a motor 906 and a link 908 connected between the flexible substrate 902 and the motor 906. The link 908 has an end fixed on the flexible substrate. The motor 906 is configured to drive the link 906 to deform the flexible substrate 902. When the link 906 is driven to retreat, the flexible substrate 902 is deformed to a concave shape as shown in FIG. 9B. The array of ultrasound transducer 904 is switched from a flat array in FIG. 9A to a concave array in FIG. 9B. The concave array can provide a smaller FOV than that of the flat array and improved resolution and penetration into an anatomy under examination.

When the link 906 is driven to extend, the flexible substrate 902 is deformed to a convex shape as shown in FIG. 9C. The array of ultrasound transducer 904 is switched from a flat array in FIG. 9A to a convex array in FIG. 9C. The convex array can provide a larger FOV than that of the flat array and scan a wider area into a human body.

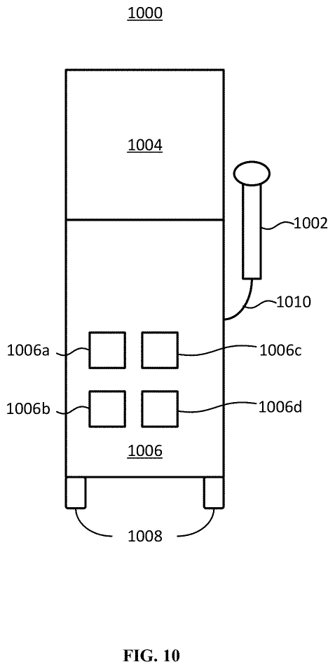

FIG. 10 is a diagram illustrating an ultrasound machine 1000, according to one example embodiment. The ultrasound machine 1000 includes one or more ultrasound probe 1002, a display device 1004, a body portion 1006, and a wheel portion 1008. The ultrasound probe 1002 is connected to the body portion 1006 via a cable 1010. The ultrasound probe 1002 includes any one of the ultrasound transducer devices disclosed herein. The ultrasound probe 1002 is configured to generate and transmit ultrasonic signals to an object under examination, and receive echo signals reflected by the object. The wheel portion 1008 includes a plurality of wheels that allow the ultrasound machine 1000 to be portable to a location of interest. The display device 1004 may be a liquid crystal display device, an organic light emitting diode display device, or any other suitable display device.

The body portion 1006 includes a controller 1006a, a memory 1006b, a battery 1006c, and a wireless communication circuit 1006d. The controller 1006 may be embodied as one or more processors configured to control the operations of the ultrasound machine 1000. For example, the controller 1006 may receive the echo signals from the ultrasound probe 1002, convert the echo signals into an image, and display the image on the display device 1004. The memory 1006b is configured to store various control instructions and ultrasound data. The battery 1006c provides power to the ultrasound machine 1000 and allows the ultrasound machine 1000 to be portable. The wireless communication circuit 1006d is configured to transmit data to and receive data from an external computer device.

In summary, the disclosed ultrasound transducer devices include a rotating transducer head that can be rotated in various axes with respect to a handle portion thus enabling different form factors as well as different scan planes. The array in the scan/transducer head may be a planar, convex, or concave array to accommodate various contours of an object under examination.

In another aspect, the disclosed ultrasound transducer devices include a head that can slide as well as slide and rotate to provide different form factors and scan planes.

In yet another aspect, the disclosed ultrasound transducer devices include a flexible array that can be configured into different shapes such as linear/planar, convex, or concave shape.

In some embodiments, the disclosed transducer array is a piezoelectric, cMUT, or pMUT array.

The foregoing description of the present disclosure has been provided for the purposes of illustration and description. It is not intended to be exhaustive or to limit the disclosure to the precise forms disclosed. The breadth and scope of the present disclosure should not be limited by any of the above-described exemplary embodiments. Many modifications and variations will be apparent to the practitioner skilled in the art. The modifications and variations include any relevant combination of the disclosed features. The embodiments were chosen and described in order to best explain the principles of the disclosure and its practical application, thereby enabling others skilled in the art to understand the disclosure for various embodiments and with various modifications that are suited to the particular use contemplated. It is intended that the scope of the disclosure be defined by the following claims and their equivalence.

What is claimed is:

1. An ultrasound transducer device comprising:
a handle portion;
a transducer head; and
a connector coupled to the transducer head and the handle portion to enable the transducer head to rotate with respect to the handle portion to provide different form factors for the ultrasound transducer device.

2. The ultrasound transducer device of claim 1, wherein the connector includes a shaft.

3. The ultrasound transducer device of claim 1, wherein the transducer head comprises a substrate and an array of ultrasound transducers disposed on the substrate.

4. The ultrasound transducer device of claim 3, wherein the ultrasound transducers are disposed on the substrate in one dimension.

5. The ultrasound transducer device of claim 4, wherein the substrate comprises a flat surface, a convex surface or a concave surface such that the array of ultrasound transducers is configured as a flat array, a convex array, or a concave array.

6. The ultrasound transducer device of claim 5, wherein the substrate is deformable such that the array of ultrasound transducers is switched among the flat array, the convex array, and the concave array.

7. The ultrasound transducer device of claim 3, wherein the ultrasound transducers are disposed on the substrate in two dimensions.

8. The ultrasound transducer device of claim 7, wherein the substrate comprises a flat surface, a convex surface or a concave surface such that the array of ultrasound transducers is configured as a flat array, a convex array, or a concave array.

9. The ultrasound transducer device of claim 8, wherein the substrate is deformable such that the array of ultrasound transducers is switched among the flat array, the convex array, and the concave array.

10. The ultrasound transducer device of claim 1, wherein the array of ultrasound transducers comprises one of a piezoelectric transducer, a capacitive micromachined ultrasound transducer, or a piezoelectric micromachined ultrasound transducers.

11. The ultrasound transducer device of claim 1, wherein the connector includes a sliding head to enable the transducer head to slide with respect to the handle portion.

12. The ultrasound transducer device of claim 11, wherein the transducer head includes a trench coupled to the sliding head of the connector such that the sliding head slides in the trench.

13. An ultrasound transducer device comprising,
a handle portion;
a transducer head; and
a connector coupled to the transducer head and the handle portion to enable the transducer head to rotate with respect to the handle portion to provide different form factors for the ultrasound transducer device,
wherein the transducer head includes a flexible substrate and an array of ultrasound transducers disposed on the flexible substrate.

14. The ultrasound transducer device of claim 13, wherein the ultrasound transducers are disposed on the flexible substrate in one dimension.

15. The ultrasound transducer device of claim 14, wherein the flexible substrate comprises a flat surface, a convex surface or a concave surface such that the array of ultrasound transducers is configured as a flat array, a convex array, or a concave array.

16. The ultrasound transducer device of claim 15, wherein the flexible substrate is deformed such that the array of ultrasound transducers is switched among the flat array, the convex array, and the concave array.

17. The ultrasound transducer device of claim 13, wherein the ultrasound transducers are disposed on the flexible substrate in two dimensions.

18. The ultrasound transducer device of claim 17, wherein the flexible substrate comprises a flat surface, a convex surface or a concave surface such that the array of ultrasound transducers is configured as a flat array, a convex array, or a concave array.

19. The ultrasound transducer device of claim 18, wherein the flexible substrate is deformed such that the array of ultrasound transducers is switched among the flat array, the convex array, and the concave array.

20. An ultrasound machine comprising:
an ultrasound transducer device configured to transmit ultrasound signals to an object and receive echo signals from the object, the ultrasound transducer device comprising:
a handle portion;
a transducer head; and
a connector coupled to the transducer head and the handle portion to enable the transducer head to rotate with respect to the handle portion to provide different form factors for the ultrasound transducer device,
a processor configured to convert the echo signals into an image; and
a display device configured to display the image.
